(19) United States
(12) Patent Application Publication
(10) Pub. No.: US 2019/0078161 A1
(43) Pub. Date: Mar. 14, 2019

(54) METHOD FOR IDENTIFYING CLINICAL TRIAL RESPONDERS FROM A PLACEBO GROUP IN MAJOR DEPRESSION

(71) Applicant: Tekada Pharmaceutical Company Limited, Osaka 541-0045, OSA (JP)

(72) Inventors: Jarlath Ffrench-Mullen, Deerfield, IL (US); Eric Lai, Deerfield, IL (US)

(73) Assignee: Tekada Pharmaceutical Company Limited, Osaka 541-0045, OSA (JP)

(21) Appl. No.: 16/085,060

(22) PCT Filed: Mar. 17, 2017
(86) PCT No.: PCT/US17/22994

§ 371 Date: Sep. 14, 2018

Related U.S. Application Data
(60) Provisional application No. 62/310,280, filed on Mar. 18, 2016.

(51) Int. Cl. C12Q 1/6883; C12Q 1/686

(52) U.S. Cl. CPC C12Q 1/6883; C12Q 1/686; C12Q 2600/158; C12Q 2600/156; C12Q 2600/16; C12Q 2600/106

(57) ABSTRACT

The present invention provides methods and kits for identifying clinical trial responders from a placebo group in clinical trials for treating depression and/or major depressive disorder (MDD). The present invention further provides methods and kits for treating depression and/or MDD in an individual, and for identifying the likelihood that an individual suffering from depression and/or MDD will respond favorably to administration of a placebo and/or experience an enhanced placebo effect when administered a placebo. These methods and kits comprise determining the presence of polymorphisms in the Brain-Derived Neurotrophic Factor gene (BDNF), the B-Cell CLL/Lymphoma 2 gene (BCL2), and/or intergenic regions in an individual.

### CROSS-REFERENCE TO RELATED APPLICATIONS

This application claims the benefits of U.S. provisional application 62/310,280, filed Mar. 18, 2016, the contents of which are incorporated herein by reference in their entirety.

### FIELD

The present invention relates to methods and kits for identifying clinical trial responders from a placebo group. The present invention also relates to methods and kits for treating depression and/or major depressive disorder (MDD) in an individual, and for identifying the likelihood that an individual suffering from depression and/or MDD will respond favorably to administration of a placebo and/or experience an enhanced placebo effect when administered a placebo. These methods and kits are based on detecting the presence of polymorphisms in the Brain-Derived Neurotrophic Factor gene (BDNF), the B-Cell CLL/Lymphoma 2 gene (BCL2), and/or intergenic regions.

### BACKGROUND

Depression is a state of low mood and aversion to activity that can affect a person's thoughts, behavior, feelings and sense of well-being. A depressed person may feel sad, anxious, empty, hopeless, worried, helpless, worthless, guilty, irritable, hurt, or restless. A number of psychiatric syndromes feature depressed mood as a main symptom. Mood disorders are a group of disorders considered to be primary disturbances of mood, such as major depressive disorder (MDD; commonly called major depression or clinical depression) where a person has at least two weeks of depressed mood or a loss of interest or pleasure in nearly all activities.

More specifically, major depressive disorder (MDD) is a disabling, severe mental disorder characterized by episodes of all-encompassing low mood accompanied by low self-esteem and loss of interest or pleasure in normally enjoyable activities. The illness tends to be chronic and repeated episodes are common. Other symptoms of MDD may include irritability or frustration, sleep disturbances, tiredness and lack of energy, changes in appetite, anxiety, agitation, restlessness, feelings of worthlessness or guilt, trouble thinking and concentrating, and unexplained physical problems, such as back pain or headaches. The disorder is a significant contributor to the global burden of disease and affects people in all communities across the world (Ferrari, PLoS Med, 10(11): e1001547 (2013)). MDD is a highly prevalent psychiatric disorder with twin studies revealing that up to 40% of MDD cases are genetically determined (Kendler, Am. J. Psychiatry, 163(1): 109-114 (2006)). Although the exact causes of MDD are unknown, it is believed that a variety of factors may be involved, such as brain chemistry and physical brain differences, hormones, inherited traits and life events.

Many types of antidepressant medications are available to treat MDD and other mood disorders that present with depression. Some available drugs include selective serotonin reuptake inhibitors (SSRIs), serotonin and norepinephrine reuptake inhibitors (SNRIs), norepinephrine and dopamine reuptake inhibitors (NDRIs), tricyclic antidepressants, monoamine oxidase inhibitors (MAOIs), and atypical antidepressants such as vortioxetine. However, despite the availability of numerous treatment options, individual response to antidepressant medication is suboptimal and variable. That is, not all individuals respond equally to a given antidepressant. As many as one half of patients do not receive adequate treatment of MDD and many respond partially or not at all to treatment.

It is believed that inherited traits may play a role in how an antidepressant affects an individual but other variables besides genetics can also affect response to medication. As a result, it is not easy to predict which medication is the best treatment option for a given patient. Accordingly, it would be beneficial to devise a method for identifying subpopulations of patients suffering from depression and/or MDD that are likely to respond most favorably to a particular MDD treatment, including treatment with or administration of a placebo.

### SUMMARY

The present invention relates to methods and kits for treating depression and/or MDD in an individual, and for identifying the likelihood that an individual suffering from depression and/or MDD will respond favorably to administration of a placebo or will experience an enhanced placebo effect in response to administration of a placebo. These methods and kits are based on the presence of polymorphisms in, for example, the BDNF gene, the BCL2 gene, and/or an intergenic region.

One aspect of the present invention provides methods for treating depression and/or MDD in an individual, comprising administering a placebo to an individual identified as (i) BDNF variant positive, (ii) BCL2 variant positive, and/or (iii) intergenic variant positive.

One aspect of the invention provides methods for identifying active agent responders in a clinical trial for treating depression and/or MDD, comprising excluding from the clinical trial an individual identified as (i) BDNF variant positive, (ii) BCL2 variant positive, and/or (iii) intergenic variant positive.

One aspect of the invention provides methods for identifying active agent responders in a clinical trial for treating depression and/or MDD, comprising excluding from data analysis data collected from an individual identified as (i) BDNF variant positive, (ii) BCL2 variant positive, and/or (iii) intergenic variant positive in the clinical trial.

In any of the aspects described herein, in some embodiments, the individual suffers from and/or has a clinical diagnosis of a major depressive disorder.

In any of the aspects described herein, in some embodiments, the individual is homozygous for a BDNF variant and/or a BCL2 variant and/or an intergenic variant. In some embodiments, the individual is heterozygous for a BDNF variant and/or a BCL2 variant and/or an intergenic variant.

In any of the aspects described herein, in some embodiments, the individual is BDNF variant positive, BCL2 variant positive, and intergenic variant positive.

In any of the aspects described herein, in some embodiments, the BDNF variant is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

In any of the aspects described herein, in some embodiments, the BCL2 variant is rs28431965.

In any of the aspects described herein, in some embodiments, the intergenic variant is rs114913258.

In any of the aspects described herein, in some embodiments, the individual has rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258 variants.

One aspect of the invention provides methods for determining the likelihood that an individual suffering from depression and/or MDD will experience an enhanced placebo effect when administered a placebo comprising: assaying a biological sample from the individual for the presence or absence of a BDNF variant and/or a BCL2 variant and/or an intergenic variant.

One aspect of the invention provides methods for determining the likelihood that an individual suffering from depression and/or MDD will respond favorably to administration of a placebo comprising: assaying a biological sample from the individual for the presence of a BDNF variant and/or a BCL2 variant and/or an intergenic variant.

In some embodiments, the sample is selected from the group consisting of a body fluid sample, a tissue sample, cells and isolated nucleic acids. In some embodiments, the isolated nucleic acids comprise DNA. In some embodiments, the isolated nucleic acids comprise RNA.

In some embodiments, the assaying comprises reverse transcribing the RNA to produce cDNA.

Some embodiments comprise detecting the presence of a BDNF variant and/or a BCL2 variant and/or an intergenic variant in nucleic acids from the individual. The individual can be, e.g., homozygous or heterozygous for the BDNF variant and/or the BCL2 variant and/or the intergenic variant.

One aspect of the invention comprises kits comprising: (i) at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405, rs28431965, and rs114913258, and (ii) a detectably labeled probe that hybridizes to the genetic variant.

In some embodiments, the kits comprise: at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405; a pair of primers that specifically hybridizes to rs28431965; and a pair of primers that specifically hybridizes to rs114913258.

### BRIEF DESCRIPTION OF THE DRAWINGS

FIG. 1 depicts a least square (LS) means plot showing Predicted Response Rates for MADRS score in the placebo arm of the TAK-315 Trial using a 24-SNP model.

FIG. 2 provides graphic results of the data showing changes from baseline in MADRS scores from the clinical TAK-315 trial after treatment with 20 mg vortioxetine and placebo using a 24-SNP model.

FIG. 3 depicts a LS means plot showing Predicted Response Rates for MADRS score in the placebo arm of the TAK-316 Trial using a 24-SNP model.

FIG. 4 provides graphic results of the data showing changes from baseline in MADRS scores from the clinical TAK-316 trial after treatment with 20 mg vortioxetine and placebo using a 24-SNP model.

FIG. 5 depicts a LS means plot showing Predicted Response Rates for MADRS score in the placebo arm of the TAK-317 Trial using a 24-SNP model.

FIG. 6 depicts a LS means plot showing Predicted Response Rates for MADRS score in the 20 mg vortioxetine and placebo arms based on combined data from the TAK-315 and TAK-316 Trials using a 24-SNP model.

FIG. 7 depicts a LS means plot showing Predicted Response Rates for MADRS score in the placebo arm based on combined data from the TAK-315 and TAK-316 Trials using a 24-SNP model.

FIG. 8 depicts a LS means plot showing Predicted Response Rates for MADRS score in the 20 mg vortioxetine and placebo arms based on combined data from the TAK-315, TAK-316, and TAK-317 Trials using a 24-SNP model.

FIG. 9 depicts a LS means plot showing Predicted Response Rates for MADRS score in the placebo arm based on combined data from the TAK-315, TAK-316, and TAK-317 Trials using a 24-SNP model.

FIG. 10 depicts a LS means plot showing Predicted Response Rates in Non-Hispanic Caucasians for MADRS score in the placebo arm based on combined data from the TAK-315, TAK-316, and TAK-317 Trials using a 24-SNP model.

FIG. 11 depicts a LS means plot showing Predicted Response Rates in African Americans for MADRS score in the placebo arm based on combined data from the TAK-315, TAK-316, and TAK-317 Trials using a 24-SNP model.

### DETAILED DESCRIPTION

Provided herein are methods and kits for identifying clinical trial responders from a placebo group. Also provided herein are methods for treating depression and/or major depressive disorder (MDD) in an individual suffering from depression or MDD. In some embodiments, the individual has been clinically diagnosed with depression and/or a depression-related mood disorder such as MDD. Also described herein are methods for identifying individuals suffering from depression and/or MDD who will likely respond favorably to administration of a placebo. Methods for identifying individuals suffering from depression and/or MDD who will likely experience an enhanced placebo effect as compared to another individual are also described.

### Target Population

The present inventors surprisingly discovered that individuals suffering from MDD who possesses a BDNF variant, a BCL2 variant, and/or an intergenic variant are more likely to experience a favorable response to a placebo than individuals who do not possess a BDNF variant, a BCL2 variant, and/or an intergenic variant. Individuals with this genotype are likely to respond favorably to administration of a placebo and/or experience an enhanced placebo effect in response to administration of a placebo.

As used herein, “BDNF” refers to the Brain-Derived Neurotrophic Factor gene, which is located on chromosome 11 [11p13; (GRCh37.p13)] in humans. The transcription start and end positions are located at 2767440-27743605 complement, respectively. An exemplary gene sequence for BDNF is NCBI Gene ID: 627, the sequence of which is incorporated by reference herein.

As used herein, “BDNF variant” is a BDNF gene with a sequence that is less than 100% identical to that of NCBI Gene ID: 627. In some embodiments, the variant has a sequence identity that is from about 75% to about 99% identical to that of NCBI Gene ID: 627, such as about 75%, about 80%, about 85%, about 90%, about 95%, or about 99% identical to that of NCBI Gene ID: 627. In some embodiments, a BDNF variant is a BDNF polynucleotide that exhibits at least one polymorphism in the BDNF coding region as compared to the coding region of NCBI Gene ID: 627. In some embodiments, a BDNF variant is associated with a favorable response to a placebo and/or an enhanced placebo effect. In some embodiments, a BDNF variant that is associated with a favorable response to a placebo and/or an enhanced placebo effect is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

An individual who is heterozygous or homozygous for a BDNF variant is “BDNF variant positive.”

As used herein, the “BCL2” gene refers to B-Cell CLL/Lymphoma 2 gene, which is located on chromosome 18 (18q21.3; GRCh37.p130. The transcription start and end positions are located at 60,790,579-60,987,011, respectively. An exemplary gene sequence for BCL2 is NCBI Gene ID: 596, the sequence of which is incorporated by reference herein.

As used herein, “BCL2 variant” is a BCL2 gene with a sequence that is less than 100% identical to that of NCBI Gene ID: 596. In some embodiments, the variant has a sequence identity that is from about 75% to about 99% identical to that of NCBI Gene ID: 596, such as about 75%, about 80%, about 85%, about 90%, about 95%, or about 99% identical to that of NCBI Gene ID: 596. In some embodiments, a BCL2 variant is a BCL2 polynucleotide that exhibits at least one polymorphism in the BDNF coding region as compared to the coding region of NCBI Gene ID: 596. In some embodiments, a BCL2 variant is associated with a favorable response to a placebo and/or an enhanced placebo effect. In some embodiments, a BCL2 variant that is associated with a favorable response to a placebo and/or an enhanced placebo effect is rs28431965.

An individual who is heterozygous or homozygous for a BCL2variant is “BCL2 variant positive.”

As used herein, “intergenic region” refers to a region between two genes. As used herein, “intergenic variant” is a region between two genes that exhibits at least one polymorphism as compared to a wild type intergenic region. In some embodiments, an intergenic variant is associated with a favorable response to a placebo and/or an enhanced placebo effect. In some embodiments, an intergenic variant that is associated with a favorable response to a placebo and/or an enhanced placebo effect is rs114913258.

An individual who is heterozygous or homozygous for an intergenic variant is “intergenic variant positive.”

As used herein, the term “variant” may include a “single nucleotide polymorphism” or “SNP”. In particular, the term “variant” may refer to the SNPs specifically disclosed herein.

As used herein, a single nucleotide polymorphism is a variation at a single position in a DNA sequence among individuals. For example, if more than 1% of a population does not carry the same nucleotide at a specific position in the DNA sequence, then this variation can be classified as a SNP. If a SNP occurs within a gene, then the gene is described as having more than one allele. In these cases, SNPs may lead to variations in the amino acid sequence. SNPs, however, are not just associated with genes; they can also occur in noncoding intergenic regions of DNA.

Although a particular SNP may not cause a disorder, some SNPs can be associated with certain diseases. These associations may allow the determination of one or more SNPs in order to evaluate an individual's genetic predisposition to develop a disease. In addition, if certain SNPs are known to be associated with a trait, stretches of DNA near these SNPs may be examined in an attempt to identify the gene or genes responsible for the trait.

Known SNPs can be taken from databases such as the SNP database at the NCBI (National Center for Biotechnology Information, Bethesda, Md.; available at ncbi.nlm.nih.gov/SNP).

The SNPs as described herein may be present on the Watson or the Crick strand, with presence of the corresponding base. If, for example, a polymorphism is present on the Watson strand as A, it is present on the Crick strand as T, if the polymorphism is present on the Watson strand as T, it is present on the Crick strand as A, if the polymorphism is present on the Watson strand as G, it is present on the Crick strand as C, and if the polymorphism is present on the Watson strand as C, it is present on the Crick strand as G, and vice versa. Also, the insertion or deletion of bases may be detected on the Watson and/or the Crick strand, with correspondence as defined above. For analytic purposes the strand identity may be defined, or fixed, or may be chosen at will, e.g. in dependence on factors such the availability of binding elements, GC-content etc. Furthermore, for the sake of accuracy, the SNP may be defined on both strands (Crick and Watson) at the same time, and accordingly be analyzed.

As used herein, an individual who suffers from MDD is an individual who meets the Diagnostic and Statistical Manual of Mental Disorders (DSM-IV-TR) criteria for MDD. In some embodiments, an individual who suffers from MDD has experienced a major depressive episode (MDE) for at least 3 months. In some embodiments, the individual has a Montgomery-Asberg Depression Rating Scale (MADRS) total score of 26, and/or a Clinical Global Impression Improvement psychological scale (CGI scale) score of 4 prior to treatment.

Depression symptoms and the degree of improvement experienced with treatment are assessed using standard depression symptom rating scales such as the Hamilton Depression Rating Scale (HAM-A), MADRS, and/or CGI scale. Treatment efficacy is determined based on an improvement in one or more depressive symptoms as measured by mean change in HAM-A total score, MADRS total score, and/or CGIS total score from baseline. A subject is determined to “respond favorably” to administration of a placebo if the placebo imparts a benefit to the subject afflicted with or diagnosed with MDD, including improvement in the condition of the subject, e.g., a human, or in one or more symptoms of the MDD. In some embodiments, a subject that responds favorably to administration of a placebo experiences a ≥50% improvement in their MADRS score in response to a placebo regimen administered to mitigate the depression as compared to their baseline score

As used herein, an individual who experiences an “enhanced placebo effect” in response to administration of a placebo experiences a greater improvement in depression symptoms when administered a placebo than an individual suffering from depression and/or MDD who has been treated with a placebo but does not possess all of the rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405 BDNF variants and/or the rs28431965 BCL2 variant and/or the rs114913258 intergenic variant.

In some embodiments, the individual resides in North America (e.g., the United States, Mexico, or Canada). In some embodiments, the individual was born in North America (e.g., the United States, Mexico, or Canada). Thus, in some embodiments, the individual is North American (e.g., American, Mexican, or Canadian).

### Methods of Treatment

In one embodiment, a method for treating depression and/or MDD in an individual identified as (i) BDNF variant positive, (ii) BCL2 variant positive, (iii) intergenic variant positive, and/or (iv) BDNF variant positive, BCL2 variant positive, and intergenic variant positive as provided herein comprises administering a placebo to the individual.

In another embodiment, a method for treating depression and/or MDD in an individual comprises determining the individual is (i) BDNF variant positive, (ii) BCL2 variant positive, (iii) intergenic variant positive, and/or (iv) BDNF variant positive, BCL2 variant positive, and intergenic variant positive and administering a placebo to the individual.

A placebo is any treatment administered to an individual that does not contain an active ingredient for treating depression and/or MDD. A placebo may be administered or ingested in any known form, such as in an oral formulation, a liquid formulation, a suspension formulation, a nasal formulation, a transdermal formulation, a rectal formulation, a topical formulation, or an injectable formulation. In some embodiments, the placebo is in the form of a tablet, a caplet, a capsule, a powder, a granule or a troche. In some embodiments, the placebo is a DBAA-el capsule, backfilled with lactose with a Swedish orange opaque color manufactured by Capsugel.

In some embodiments, a placebo is administered or ingested for at least 5, 6, 7, or 8 weeks. In some embodiments, a placebo is administered one or more times per day. In some embodiments, a placebo is administered one or more times per week.

Some embodiments comprise methods for determining the likelihood that an individual suffering from depression and/or MDD is likely to experience an enhanced placebo effect when administered a placebo. In some embodiments, the methods comprise assaying a sample from the individual suffering from depression and/or MDD to determine the presence or absence of a BDNF variant and/or a BCL2 variant and/or an intergenic variant in nucleic acids from the individual. The individual is determined to be likely to respond favborably to administration of a placebo and/or experience an enhanced placebo effect when administered a placebo if a BDNF variant and/or a BCL2 variant and/or an intergenic variant are present in nucleic acids from the individual.

### Methods of Predicting Response to a Placebo

Methods for determining the likelihood that an individual suffering from depression and/or MDD will respond favorably to administration of a placebo is also provided herein. In some embodiments, the methods comprise assaying a sample from the individual to determine the presence of a BDNF variant and/or a BCL2 variant and/or an intergenic variant in nucleic acids from the individual, and determining that the individual is likely to respond favorably to administration of a placebo when the individual is homozygous or heterozygous for a BDNF variant and/or a BCL2 variant and/or an intergenic variant.

In some embodiments, the methods comprise administering a placebo to the BDNF variant positive, BCL2 variant positive, and/or intergenic variant positive individual.

In some embodiments, the methods comprise assaying a sample from the individual to determine the presence or absence of a BDNF variant and/or a BCL2 variant and/or an intergenic variant in nucleic acids from the individual, and determining that the individual is likely to respond favorably to administration of a placebo when the individual possesses a BDNF variant and/or a BCL2 variant and/or an intergenic variant.

In some embodiments, determining whether an individual is BDNF variant positive, BCL2 variant positive, and/or intergenic variant positive involves obtaining a biological sample from an individual. The biological sample can be any substance that contains nucleic acids from the individual, such as a body fluid sample, a tissue sample, a stool sample, cells from the individual, and/or isolated nucleic acids from the individual. Exemplary body fluid samples include blood, plasma, serum, cerebrospinal fluid, bile, and saliva. Exemplary tissue samples include tissue biopsy samples. Exemplary cell samples include buccal swabs or cells obtained from biological samples taken from the individual. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

As used herein, “isolated nucleic acids” means nucleic acid that are removed to at least some extent from the cellular material from which they originated. However, “isolated” does not require that nucleic acid be completely pure and free of any other components. Examples of isolated nucleic acid are those obtained using commercial nucleic acid extraction kits.

In some embodiments, a sample from an individual contains DNA and/or RNA from the individual. In some embodiments, assaying a sample involves extracting nucleic acids from a biological sample to determine that the individual is positive for any of the variants described herein. For example, some embodiments comprise extracting nucleic acids from a biological sample to determine that the individual is BDNF variant positive and/or BCL2 variant positive and/or intergenic variant positive. Various methods of extraction are suitable for isolating DNA or RNA. Suitable methods include phenol and chloroform extraction. See Maniatis et al., Molecular Cloning, A Laboratory Manual, 2d, Cold Spring Harbor Laboratory Press, pages 16-54 (1989). Numerous commercial kits also yield DNA and/or RNA. However, nucleic acid extraction is not essential and a sample, such as blood or saliva, may be assayed directly to determine that the individual is BDNF variant positive and/or BCL2 variant positive and/or intergenic variant positive without extracting nucleic acids from the sample.

In some embodiments, assaying a sample comprises reverse transcribing RNA to produce cDNA.

In some embodiments, assaying a sample comprises amplifying nucleic acids in the sample or nucleic acids derived from nucleic acids in the sample (e.g. cDNA). Amplification methods which may be used include variations of RT-PCR, including quantitative RT-PCR, for example as adapted to the method described by Wang, A. M. et al., Proc. Natl. Acad. Sci. USA 86:9717-9721, (1989), or by Karet, F. E., et al., Analytical Biochemistry 220:384-390, (1994). Another method of nucleic acid amplification or mutation detection which may be used is ligase chain reaction (LCR), as described by Wiedmann, et al., PCR Methods Appl. 3:551-564, (1994). An alternative method of amplification or mutation detection is allele specific PCR (ASPCR). ASPCR which utilizes matching or mismatching between the template and the 3′ end base of a primer well known in the art. See e.g., U.S. Pat. No. 5,639,611, which is incorporated herein by reference and made a part hereof. In some embodiments, amplification is conducted using a platform from NuGEN, Inc., such as an Ovation® (e.g., RNA Amplication System, FFPE WTA System, Pico WTA System V2, RNA-Seq System V2, Ultraflow Multiplex System) or Encore® (e.g., 384 Multiplex System 1A-D) platform.

A person skilled in the art will recognize that, based on the SNP and associated sequence information disclosed herein, detection reagents can be developed and used to assay any SNP of the present technology individually or in combination, and that such detection reagents can be incorporated into a kit.

The term “kit” as used herein in the context of SNP detection reagents refers to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.).

Accordingly, the present technology further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs described herein. The kits can optionally include various electronic hardware components. For example, arrays (“DNA chips”) and microfluidic systems (“lab-on-a-chip” systems) provided by various manufacturers typically comprise hardware components. Some kits (e.g., TaqMan probe/primer sets) may not include electronic hardware components, but may be comprised of, for example, one or more SNP detection reagents (along with other optional biochemical reagents) packaged in one or more containers.

In some embodiments, a SNP detection kit contains one or more detection reagents and other components (e.g., buffers, reagents, enzymes having polymerase activity, enzymes having polymerase activity and lacking 5′→3′ exonuclease activity or both 5′→3′ and 3′→5′ exonuclease activity, ligases, enzyme cofactors such as magnesium or manganese, salts, chain extension nucleotides such as deoxynucleoside triphosphates (dNTPs) or biotinylated dNTPs, and in the case of Sanger-type DNA sequencing reactions, chain terminating nucleotides (i.e., dideoxynucleoside triphosphates (ddNTPs), positive control sequences, negative control sequences, and the like) to carry out an assay or reaction, such as amplification and/or detection of a SNP-containing nucleic acid molecule. In some embodiments, a kit contains a means for determining the amount of a target nucleic acid, determining whether an individual is heterozygous or homozygous for a polymorphism, detecting a gene transcript, and/or comparing the amount with a standard. In some embodiments, the kit comprises instructions for using the kit to detect the SNP-containing nucleic acid molecule of interest. In some embodiments, the kits contain reagents to carry out one or more assays to detect one or more SNPs disclosed herein. In some embodiments, SNP detection kits are in the form of nucleic acid arrays or compartmentalized kits, including microfluidic/lab-on-a-chip systems.

The kits may further comprise one or more of: wash buffers and/or reagents, hybridization buffers and/or reagents, labeling buffers and/or reagents, and detection means. The buffers and/or reagents can be optimized for the particular amplification/detection technique for which the kit is intended. Protocols for using these buffers and reagents for performing different steps of the procedure may also be included in the kit.

In some embodiments, the SNP detection kits comprise at least one set of primers (e.g., comprising one matched allele-specific primer and one mismatched allele-specific primer) and, optionally, a non-extendable oligonucleotide probe. Each kit can comprise reagents that render the procedure specific. Thus, a kit intended to be used for the detection of a particular SNP can comprise a matched and mismatched allele-specific primers set specific for the detection of that particular SNP, and optionally, a non-extendable oligonucleotide probe. A kit intended to be used for the multiplex detection of a plurality of SNPs comprises a plurality of primer sets, each set specific for the detection of one particular SNP, and, optionally, a plurality of corresponding non-extendable oligonucleotide probes.

In some embodiments, the SNP detection kits comprise multiple pairs of primers for one or more target SNP loci, wherein said primers are designed so that the lengths of said PCR products from different SNP loci or from different alleles of the same SNP locus are sufficiently distinguishable from each other in capillary electrophoresis analysis, thus making them suitable to multiplex PCR. The SNP detection kit can further comprise a fluorescently labeled single-base extension/termination reagent, i.e., ddNTPs, to label the primers during the multiplex PCR reaction (e.g., SNaPshot Multiplex). The chemistry of the SNP detection kit can be based on the dideoxy single-base extension of the unlabeled primers. Each primer can bind to its target SNP loci in the presence of fluorescently labeled ddNTPs and the polymerase extends the primer by one nucleotide, adding a single ddNTP to its 3′ end. The identity of the incorporated nucleotide can be determined by the fluorescence color readout. In some embodiments, the kits comprise multiple pairs of primers for simultaneously detecting at least one SNP locus having two or more different alleles. In some embodiments, the kits comprise multiple pairs of primers for simultaneously detecting different genotypes among 1-8 different SNP loci. In some embodiments, the SNP detection kit comprises multiple pairs of primers that have the annealing temperatures designed to be used in a single amplification reaction. In some embodiments, the kits further comprise an internal control polynucleotide and/or multiple control primers for conducting multiplex PCR using the internal control polynucleotide as a template.

In some embodiments, SNP detection kits may contain, for example, one or more probes, or pairs of probes, that hybridize to a nucleic acid molecule at or near each target SNP position. Multiple pairs of allele-specific probes may be included in the kit to simultaneously assay multiple SNPs, at least one of which is a SNP disclosed herein. In certain embodiments, multiple pairs of allele-specific probes are included in the kit to simultaneously assay all of the SNPs described herein. In some embodiments, the kit includes capture primers and optionally extension primers for the detection of one or a plurality of SNPs of one or more intergenic regions and/or genes selected from the group consisting of BDNF and BCL2.

In some embodiments, the SNP detection kits comprise at least one set of pre-selected nucleic acid sequences that act as capture probes for the extension products. The pre-selected nucleic acid sequences (allele-specific probes) may be immobilized on an array or beads (e.g., coded beads), and can be used to detect at least 1, 4, 10, 11, 24, all, or any combination of the SNPs disclosed herein. By way of example only, the kits may include polystyrene microspheres that are internally dyed with two spectrally distinct fluorescent dyes (e.g., x-MAP™ microbeads, Luminex Corp. (Austin, Tex.)). Using precise ratios of these fluorophores, a large number of different fluorescent bead sets can be produced (e.g., a set of 100). Each set of beads can be distinguished by its code (or spectral signature) and can be used to detect a large number of different extension products in a single reaction vessel. These sets of fluorescent beads with distinguishable codes can be used to label extension products. Labeling (or attachment) of extension products to beads can be by any suitable means including, but not limited to, chemical or affinity capture, cross-linking, electrostatic attachment, and the like. In some embodiments, labeling of extension products is carried out through hybridization of the allele-specific primers and the tag probe sequences. The magnitude of the biomolecular interaction that occurs at the microsphere surface is measured using a third fluorochrome that acts as a reporter (e.g., biotinylated dNTPs). Because each of the different extension products is uniquely labeled with a fluorescent bead, the captured extension product (indicative of one allele of a SNP of interest) can be distinguishable from other different extension products (including extension products indicative of other alleles of the same SNP and extension products indicative of other SNPs of interest). Following hybridization, the microbeads can be analyzed using methods such as flow cytometry. In embodiments where the primer extension reaction is carried out in the presence of biotinylated dNTPs, the reaction between beads and extension products may be quantified by fluorescence after reaction with fluorescently-labeled streptavidin (e.g., Cy5-streptavidin conjugate) using instruments such as the LUMINEX® 100™ Total System, LUMINEX® 100™ IS Total System, LUMINEX™ High Throughput Screening System).

Some embodiments provide methods of identifying the SNPs disclosed herein in a biological sample comprising incubating a test sample of nucleic acids obtained from the subject with an array comprising one or more probes corresponding to at least one SNP position disclosed herein, and assaying for binding of a nucleic acid from the test sample with one or more of the probes. Conditions for incubating a test sample with a SNP detection reagent from a kit that employs one or more such SNP detection reagents can vary. Incubation conditions depend on factors such as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

In some embodiments, the SNP detection kits of the present technology include control analytes for spiking into a sample, buffers, including binding, washing and elution buffers, solid supports, such as beads, protein A or G or avidin coated sepharose or agarose, etc., and a matrix-assisted laser desorption/ionization (MALDI) sample plate. The kit may also contain a database, which may be a table, on paper or in electronic media, containing information for one or a plurality of SNPs of intergenic regions and/or one or more genes selected from the group consisting of BDNF and BCL2. In some embodiments, the kits contain programming to allow a robotic system to perform the present methods, e.g., programming for instructing a robotic pipettor or a contact or inkjet printer to add, mix and remove reagents. The various components of the kit may be present in separate containers or certain compatible components may be precombined into a single container, as desired.

In some embodiments, the kits include one or more other reagents for preparing or processing an analyte sample for matrix-assisted laser desorption/ionization time-of-flight mass spectrometry (MALDI-TOF). The reagents may include one or more matrices, solvents, sample preparation reagents, buffers, desalting reagents, enzymatic reagents, denaturing reagents, where calibration standards such as positive and negative controls may be provided as well. As such, the kits may include one or more containers such as vials or bottles, with each container containing a separate component for carrying out a sample processing or preparing step and/or for carrying out one or more steps of a MALDI-TOF protocol.

In addition to above-mentioned components, the kits can include instructions for using the components of the kit, e.g., to prepare a MALDI-TOF sample plate and/or assess a sample. The instructions, such as for preparing or assessing a sample via MALDI-TOF, are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate. In addition to the database, programming and instructions, the kits may also include one or more control analyte mixtures, e.g., two or more control samples for use in testing the kit.

In some embodiments, the methods comprise determining the presence of a genetic variant with nucleic acid sequencing. Sequencing can be performed using any number of methods, kits or systems known in the art. One example is using dye terminator chemistry and an ABI sequencer (Applied Biosystems, Foster City, Calif.). Sequencing also may involve single base determination methods such as single nucleotide primer extension (“SNAPSHOT®” sequencing method) or allele or mutation specific PCR. The SNAPSHOT® Multiplex System is a primer extension-based method that enables multiplexing up to 10 SNPs (single nucleotide polymorphisms). The chemistry is based on the dideoxy single-base extension of an unlabeled oligonucleotide primer (or primers). Each primer binds to a complementary template in the presence of fluorescently labeled ddNTPs and AMPLITAQ® DNA Polymerase, FS. The polymerase extends the primer by one nucleotide, adding a single ddNTP to its 3′ end. SNAPSHOT® Multiplex System is commercially available (ABI PRISM. SNAPSHOT® Multiplex kit, Applied Biosystems Foster City, Calif.). Products generated using the ABI PRISM® SNaPshot® Multiplex kit can be analyzed with GENESCAN® Analysis Software version 3.1 or higher using ABI PRISM® 310 Genetic Analyzer, ABI PRISM® 3100 Genetic Analyzer or ABI PRISM® 3700 DNA Analyzer.

Next generation sequencing (NGS) may be used to determine an individual's genotype. Next generation sequencing is a high throughput, massively parallel sequencing method that can generate multiple sequencing reactions of clonally amplified molecules and of single nucleic acid molecules in parallel. This allows increased throughput and yield of data. NGS methods include, for example, sequencing-by-synthesis using reversible dye terminators, and sequencing-by-ligation. Non-limiting examples of commonly used NGS platforms include Miseq/Nextseq/HiSeq (Illumina, Inc.), ROCHE 454™ GS FLX™-Titanium (Roche Diagnostics), XMAP® (Luminex Corp.), IONTORRENT™ (Life Technologies Corp.) ABI SOLiD™ System (Applied Biosystems, Foster City, Calif.), OVATION® (NuGEN, Inc.), ENCORE® (NuGEN, Inc.), and Mondrian™ (NuGEN, Inc.).

Some embodiments as described herein are directed to kits comprising: (i) at least one pair of primers that specifically hybridizes to a genetic variant as described herein, and (ii) a detectably labeled probe that hybridizes to the genetic variant. In some embodiments, the kits comprise at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258.

Some embodiments are directed to kits comprising: (i) at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs 28431965, and rs114913258, and (ii) a detectably labeled probe that hybridizes to the genetic variant.

Some embodiments are directed to kits comprising: at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405; a pair of primers that specifically hybridizes to rs28431965; and a pair of primers that specifically hybridizes to rs114913258.

### Method for Identifying Clinical Trial Responders

Some embodiments comprise identifying active agent responders in a clinical trial for treating depression and/or MDD. In some embodiments, the methods comprise improving the accuracy of clinical data for treating depression and/or MDD. In some embodiments, the methods comprise excluding an individual that possesses a BDNF variant and/or a BCL2 variant and/or an intergenic variant from a clinical trial for depression and/or MDD. For instance, some methods comprise excluding an individual that possesses rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405 BDNF variants and/or the rs28431965 BCL2 variant and/or the rs114913258 intergenic variant from a clinical trial for depression and/or MDD.

In other embodiments, methods as described herein comprise accounting for individuals that possess a BDNF variant and/or a BCL2 variant and/or an intergenic variant when analyzing data from a clinical trial for depression and/or MDD. For instance, in some embodiments, data collected from an individual that possesses a BDNF variant and/or a BCL2 variant and/or an intergenic variant is excluded when data is used to identify active agent responders. In particular embodiments, data collected from an individual that possesses rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405 BDNF variants and/or the rs2843195 BCL2 variant and/or the rs114913258 intergenic variant is excluded when data is used to identify active agent responders.

In some embodiments, methods as described herein comprise placing individuals that possess a BDNF variant and/or a BCL2 variant and/or an intergenic variant into a first arm of a clinical trial for depression and/or MDD. For instance, in some embodiments individuals that possess rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405 BDNF variants and/or the rs2843195 BCL2 variant and/or the rs114913258 intergenic variant are placed into a first arm of a clinical trial for depression and/or MDD.

In some embodiments, individuals that do not possess the rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405 BDNF variants and/or the rs2843195 BCL2 variant and/or the rs114913258 intergenic variant are placed into a second arm of a clinical trial for depression and/or MDD.

### EXAMPLES

### Example 1—MDD Trials

### Study Design—Treatment Groups

Multicenter, randomized, double-blind, parallel-group, placebo-controlled, drug-referenced, fixed-dose studies were conducted to evaluate the efficacy and safety of vortioxetine in the acute treatment of adult patients with MDD. A total of 595 individuals meeting the diagnostic criteria from the DSM-IV-TR for recurrent MDD were included in the studies, TAK-315, -316 and -317. The current major depressive disorder for each individual was confirmed by the Structured Clinical Interview for DSM Disorders (SCID). The individuals had a reported duration of their current MDE of at least 3 months. The individuals also had a total MADRS score of 26 and a CGI-S score of at the screening and baseline visits. Individuals were treated with 10, 15 or 20 mg vortioxetine, or a different drug, or administered a placebo daily for 8 weeks.

Subjects were seen weekly during the first 2 weeks of treatment and then every 2 weeks up to the end of the 8-week treatment period. The primary outcome measure was change from baseline in MADRS total score after 8 Weeks of treatment. MADRS is a depression rating scale consisting of 10 items, each rated 0 (no symptom) to 6 (severe symptom). The 10 items represent the core symptoms of depressive illness. The rating is based on a clinical interview with the patient, moving from broadly phrased questions about symptoms to more detailed ones, which allow a precise rating of severity, covering the most recent 7 days. Total score is from 0 to 60, with a higher the score being the more severe.

Secondary outcome measures included the proportion of responders at week 8 (responders defined as a 50% decrease in MADRS total score from baseline); a change from baseline in MADRS total score at week 8; and a change in clinical status using CGI-I score at week 8. The CGI-I scale is a 7-point scale rated from 1 (very much improved) to 7 (very much worse). The investigator rated the patient's overall improvement relative to baseline, whether or not, in the opinion of the investigator, this was entirely due to the treatment.

### Study Design—Genotype Determination

Nucleic acid samples from the individuals were run on an ILLUMINA™ HumanOmni5EXOME whole genome bead-chip array according to the manufacturer's protocol. The raw dataset of 595 samples times 4,641,218 variants was narrowed to 572 samples times 3,923,897 variants following quality control (QC). The placebo arm of dataset included 335 samples.

### Association Testing of Genomic Features

The following types of genomic features based on genetic variants (e.g., single nucleotide polymorphisms, i.e., SNPs, insertions, or deletions) were considered in the present study: (i) A SNP with Minor Allele Frequency (MAF)≥5% (i.e., a single genetic variant); and (ii) a gene of interest, which was defined as the region between the transcription start and end positions plus 5 kb upstream and downstream of the transcription start and end positions.

For all single variant analyses, a genotypic or dominant model was considered for variants that passed genotypic QC. Specifically, the following genetic models were used to accommodate single variant analyses: (i) A genotypic model (i.e. a model with g−1 degrees of freedom (d.f.), where g=the number of observed genotypes) was considered for common variants (i.e. MAF≥5%) with genotype counts of at least 5 for all genotype categories; and (ii) a dominant model (i.e. a model with 1 d.f, where presence vs. absence of the minor allele was modeled) was used for (a) common variants with less than 5 observations for any of the genotypes and (b) less common (i.e., 1%<MAF<5%) or rare (i.e., MAF<1%) variants with at least 5 observations in the heterozygous and rare homozygous groups combined. A variant was omitted from single variant testing if the variant had less than 5 observations in the heterozygous and rare homozygous groups combined. For multi-variant analyses, each variant was coded as the number of minor alleles (i.e., 0, 1, or 2).

A tiered approach was employed to prioritize the genomic regions of interest in association testing. Tier 1 genomic regions were defined in part as genes/SNPs residing in regions covered by the Illumina™ HumanOmni5EXOME whole genome bead-chip array. Region-based testing and single variant testing were performed using a tiered analysis approach, where: tier 1 included 11 genes/variants from dbGaP analysis (National Institutes of Health (HIH) database of archived genotypes and phenotypes of studies that have investigated the interaction of genotype and phenotype) and prior knowledge; tier 2 included 87 genes/variants from MDD risk genes reported in literature; and tier 3 included remaining genes/variants in the human genome. Multiplicity adjustment was performed using Bonferroni adjustment and alpha-levels of 0.05 for tiers ½ and 0.1 for tier 3 genes.

A main effect model was utilized to identify genes or single variants prognostic of response using the placebo samples. The outcomes of interest included: (i) Primary outcome: Response/non-response defined as patients who had a ≥50% decrease in MADRS total score at week 8 using last post-baseline observation carried forward (LOCF) and (ii) Secondary outcome: Change from baseline in MADRS total score.

The effect of genetic variation (single variant or gene-level testing) on the outcome of interest was assessed within placebo treated subjects. Multiplicity adjustment was considered by tier (Tiers 1, 2 and 3) for each outcome (e.g. MADRS response; change of MADRS). The discovery of the placebo genetic signature was mainly based on the results of the primary outcome.

A flexible regression framework was considered for the outcome of MADRS response within the placebo arm, given by:

logit(π(Xᵢ,Bᵢ,Tᵢ))=Xᵢ^{T}α+Tᵢβ_{T}+f(Bᵢ) (1)

where π denotes the probability of response, Xᵢ and Bᵢ were the ith subject's covariate vector and biomarker vector, Tᵢ denotes an indicator variable, and a was the coefficient vector for the covariate vector. The top 3 Principal Components derived from a Principal Component Analysis as well as trial, age, gender, smoking status, and alcohol usage were included in the model as covariates. The model (1) was also adapted for continuous outcomes (e.g., change of MADRS) by replacing the logistic model with the linear model. The biomarker vector Bᵢ was a general placeholder that represented the corresponding biomarker.

### Subgroup Identification

Prognostic subgroup identification was performed within the placebo arm (N=335). Subgroup identification was considered for the primary endpoint MADRS response. A subgroup [symbol] was assumed to exhibit better response as measured by the MADRS response among the placebo samples. The problem of subgroup identification was written in the logistic regression framework as

logit(π(Xᵢ,Gᵢ))=Xᵢ^{T}α₀+βₛ·I(i∈ [symbol] )

where βₛ denoted placebo effect for subgroup [symbol] . Since true subgroup membership cannot be observed, biomarkers were used as surrogates to infer subgroup membership.

A multi-marker composite score approach was used in the development of a genetic signature that could be used to infer subgroup membership. A subgroup was identified using a two-stage approach: Stage 1—Develop a composite score using a subset of biomarkers (i.e. genetic variants) via a penalized regression approach; and Stage 2—Find a composite score cutoff that defines a subgroup. The composite score was derived by fitting a working model

logit(π(Xᵢ,Gᵢ))=Xᵢ^{T}α₀+Bᵢ^{T}θ

where Bᵢ^{T}θ contained main effect terms of the biomarkers. The composite score was then defined as γ=Bᵢ^{T}θ, in which increasing values of the composite score would be indicative of a benefit (i.e., increasing Odds Ratio). Since it was not expected that all biomarkers were important to define subgroup membership, the model above was fit using the elastic net (Zou and Hastie, J. R. Statist. Soc. B, 67: 301-320 (2005)), which inherently performed feature selection through penalized regression. For biomarkers that did not contribute significantly to the composite score, the corresponding parameter estimates could be shrunk to zero in the composite score calculation and subsequently only biomarkers with non-zero parameter estimates were used in the definition of a genetic signature.

A subgroup of patients was defined using the estimated composite scores {\hat{γ}ᵢ: i=1, . . . , n} for n patients. Given a threshold of τ∈{\hat{γ}=1, . . . n; Tᵢ=1}, the subgroup was defined as S_{τ}={i: \hat{γ}ᵢ≥τ, i=1, . . . , n}. To choose the optimal value τ=τ_{*} and the corresponding subgroup S_{τ}_{_{*}} a grid search was used to consider all possible τ's in the designated range of 25-75% and to choose the optimal value τ=τ; maximizing a χ²-test statistic. To account for the multiple testing performed when searching for the optimal threshold, significance of the subgroup main effect at the optimal cutoff was evaluated using a parametric bootstrap approach.

### Example 2-24—SNP Model

The subgroup showing enhanced placebo effect was identified using a combined elastic net/bootstrapping approach, similar to that set forth in Li et al., The Pharmacogenomics Journal, 14(5): 439-45 (2014), which is incorporated herein by reference and made a part hereof

Table 1 shows genes and gene combinations whose expression levels can be combined in multigene models that significantly correlate with enhanced placebo effect in patients administered a placebo.

TABLE 1
24 variants used in genetic signature

| | | | B | A | G = 0 | G = 1 | G = 2 |
|---|---|---|---|---|---|---|---|
| rs # | Coefficient | Bootstrap CI (95%) | Allele | Allele | (AA) | (AB) | (BB) |
| rs7124442 | 0.1809 | (0.000, 1.306) | C | T | TT | TC | CC |
| rs76327806 | 0.4244 | (0.000, 3.993) | C | T | TT | TC | CC |
| rs6265 | −0.0030 | (−1.568, 0.048) | T | C | CC | CT | TT |
| rs12273539 | −0.0017 | (−1.133, 0.000) | T | C | CC | CT | TT |
| rs11030104 | 0.0551 | (0.000, 2.034) | G | A | AA | AG | GG |
| rs12291186 | −0.1257 | (−1.397, 0.719) | C | A | AA | AC | CC |
| rs55848362 | 0.3375 | (0.000, 1.618) | T | C | CC | CT | TT |
| rs72878196 | 0.0458 | (−1.41E−05, 0.466) | C | A | AA | AC | CC |
| rs10835211 | 0.3838 | (0.000, 2.309) | A | G | GG | GA | AA |
| rs16917237 | 0.1721 | (0.000, 2.147) | T | G | GG | GT | TT |
| rs73446388 | −0.2612 | (−2.593, 0.000) | T | C | CC | CT | TT |
| rs12293082 | 0.0966 | (0.000, 1.651) | C | T | TT | TC | CC |
| rs4923468 | 0.0459 | (0.000, 0.464) | A | C | CC | CA | AA |
| rs11030119 | −0.7055 | (−2.643, −0.249) | A | G | GG | GA | AA |
| rs76368953 | 0.1402 | (−0.351, 1.425) | G | A | AA | AG | GG |
| rs72881263 | 0.0463 | (0.000, 0.464) | G | A | AA | AG | GG |
| rs80083564 | 0.2426 | (−0.620, 1.299) | T | G | GG | GT | TT |
| rs74435097 | −0.0111 | (−2.321, 0.000) | G | A | AA | AG | GG |
| rs10219241 | 0.0505 | (0.000, 0.521) | A | G | GG | GA | AA |
| rs80128513 | −0.0088 | (−1.525, 0.593) | A | C | CC | CA | AA |
| rs28383487 | −0.7775 | (−3.276, −0.238) | T | G | GG | GT | TT |
| rs55958405 | 0.6898 | (0.000, 2.062) | A | C | CC | CA | AA |
| rs28431965 | 0.8550 | (0.000, 2.585) | C | T | TT | TC | CC |
| rs114913258 | 0.7526 | (0.000, 3.656) | T | C | CC | CT | TT |

Table 2 provides General information on the 24 SNPs listed in Table 1. In Table 2, the column descriptions are as follows: Gene: gene name; RS#: rs number in dbSNP; Chr: chromosome; Position: physical position (using hg19 coordinates); MAF: Minor Allele Frequency, calculated using 535 after-QC samples in the 20 mg and placebo arms.

TABLE 2
General Information on SNPs in 24-SNP Model

| rs # | Gene | Chr | Position | MAF |
|---|---|---|---|---|
| rs7124442 | BDNF | 11 | 27677041 | 0.304 |
| rs76327806 | BDNF | 11 | 27678411 | 0.024 |
| rs6265 | BDNF | 11 | 27679916 | 0.159 |
| rs12273539 | BDNF | 11 | 27683311 | 0.102 |
| rs11030104 | BDNF | 11 | 27684517 | 0.177 |
| rs12291186 | BDNF | 11 | 27696318 | 0.107 |
| rs55848362 | BDNF | 11 | 27698596 | 0.04 |
| rs72878196 | BDNF | 11 | 27701025 | 0.042 |
| rs10835211 | BDNF | 11 | 27701365 | 0.193 |
| rs16917237 | BDNF | 11 | 27702383 | 0.174 |
| rs73446388 | BDNF | 11 | 27721621 | 0.02 |
| rs12293082 | BDNF | 11 | 27725504 | 0.036 |
| rs4923468 | BDNF | 11 | 27725775 | 0.042 |
| rs11030119 | BDNF | 11 | 27728102 | 0.266 |
| rs76368953 | BDNF | 11 | 27731430 | 0.02 |
| rs72881263 | BDNF | 11 | 27732335 | 0.042 |
| rs80083564 | BDNF | 11 | 27733143 | 0.093 |
| rs74435097 | BDNF | 11 | 27736281 | 0.044 |
| rs10219241 | BDNF | 11 | 27737123 | 0.472 |
| rs80128513 | BDNF | 11 | 27742122 | 0.065 |
| rs28383487 | BDNF | 11 | 27743556 | 0.014 |
| rs55958405 | BDNF | 11 | 27745683 | 0.017 |
| rs28431965 | BCL2 | 18 | 60947535 | 0.034 |
| rs114913258 | Intergenic | 1 | 20760566 | 0.01 |

Table 3 shows flanking DNA sequences of the SNPs used in the 24-SNP model.

TABLE 3
Flanking DNA Sequences of SNPs in 24-SNP Model

| | | SEQ ID |
|---|---|---|
| rs # | Flanking Sequence | NO |
| rs7124442 | AAGGAAGCTGCATAAAGTTGACATA[T/C]AGCAGATATTCC | 1 |
| | AAGCATTCCTTAC | |
| rs76327806 | ATACGAGTGTCATGATGTGACACAA[T/C]GTGTTCACTTGTT | 2 |
| | CACAGCAGTGGT | |
| rs6265 | TCCTCATCCAACAGCTCTTCTATCA[T/C]GTGTTCGAAAGTGT | 3 |
| | CAGCCAATGAT | |
| rs12273539 | ACTCAATGCTTCATCACTTCTGCTC[T/C]GATCAGGACAGAG | 4 |
| | TCCTTGGAGTGC | |
| rs11030104 | ATTAAAAAGCAGATAACACTACCAC[A/G]TACTAACTGTCCT | 5 |
| | ACAATTTCCTGT | |
| rs12291186 | AGAAGACATGCAAACTCAAATACCA[A/C]CATATTTCATCTA | 6 |
| | AGCATAGGACAG | |
| rs55848362 | ACTAGAGCTGGCGCAAGCCCATGGC[T/C]ATGGTGAGGCAG | 7 |
| | CGTTTCCACTGGA | |
| rs72878196 | CTTTGCTTTAGGAAAATCATTTTCA[A/C]GGTTGCTTCATGCA | 8 |
| | AAGTGGGAATA | |
| rs10835211 | TTCCTGTTTCACCAGCAGAGCTCTG[A/G]TCGCTCAGTTGAA | 9 |
| | GCTGAAAGTCAG | |
| rs16917237 | TTCCTACCACCATTACATACTTCTG[T/G]TAATCAATTATCTT | 10 |
| | CCTTCTCCCCT | |
| rs73446388 | AGGAAAGAAGGAGACTGGCCTCGTC[T/C]CACAACTTTGGG | 11 |
| | GTGGGGGATCCCC | |
| rs12293082 | AATTGTACCAGTGCCTTGCATGGAA[T/C]TCTAAATATTATT | 12 |
| | TTTTATTTGCTT | |
| rs4923468 | CTTGAAAAAATAATGCTACCTATTT[A/C]ACTTTTGTGGGGC | 13 |
| | TTAAAAATTAAT | |
| rs11030119 | TTAAGTCACCACTCAGACTTTTCTC[A/G]TAGCAAAAGATCA | 14 |
| | GATCTCACAACC | |
| rs76368953 | GAGATGATTTAGGTGATTTCAATGC[A/G]TGAAGGGAATCA | 15 |
| | GTGAACTAGATCA | |
| rs72881263 | CAAACTTATGAAATAGCTTCTAAAC[A/G]TACTAAATCCTAA | 16 |
| | TCCAAACAATTT | |
| rs80083564 | AGATGGGGAGGCACAACTTCATAAT[T/G]GACTTTGGAACC | 17 |
| | AGAAAACTTTGGG | |
| rs74435097 | CTTAATTTCCCCTACACTGCAAGAA[A/G]TAGTTATAGACTA | 18 |
| | TATCTTTTATAT | |
| rs10219241 | AACTTTCCATTAGGCCTGAAATTTT[A/G]AAAACTATTTGTA | 19 |
| | AAAAATGTTTTT | |
| rs80128513 | CTCCAGCCCCGATCTCAGTGTGAGC[A/C]GAACCTCAGAAA | 20 |
| | AGACGCTTTTTAA | |
| rs28383487 | CTTGTCAGGCTAGGGCGGGAAGACC[T/G]CTGGGGAACTTG | 21 |
| | TTGCTTATCAGCG | |
| rs55958405 | TATATTTTTCTGACTCTTTTGTCAA[A/C]AGTTTGTGGTCTGT | 22 |
| | ATAGAAACTAT | |
| rs28431965 | CTTTCAAAGCCTCAGTTTCCGTATC[T/C]ATTAAATGAAGGT | 23 |
| | GATAATATCTAT | |
| rs114913258 | TCTGTCTCCTCTGCCAGTCTGAGAG[T/C]TCCTCTGGGACAG | 24 |
| | GGTACTTGGTCT | |

The 24 variants for subgroup identification based on association testing includes: (i) variants from gene BDNF (61 variants in the gene that were on Omni5Exome and passed genotypic quality control); (ii) variant rs28431965 from gene BCL2; and (iii) intergenic variant rs114913258 which is 5 kb upstream of UCSC gene LOC339505. This set of variants (63 variants) was considered by the subgroup identification approach using 335 patients in the placebo arms of trials 315, 316, and 317. The results suggested that 24 variants significantly defined a subgroup within the placebo arm that showed statistical evidence of higher MADRS response. With the genetic signature defined by the SNP model, a patient's score is defined by

$${Score}_{i} = {\sum }_{j} {coefficient}_{j} \times ( \# B Alleles of SNP j )$$

and the patient's subgroup membership is defined by

$${Membership}_{i} = \{ \begin{matrix}1 , & if {Score}_{i} \geq threshold \\ 0 , & if {Score}_{i} < threshold\end{matrix}$$

Specifically, the formula is expressed as follows, with a threshold of r=0.0618

$$score = 0.1809 * rs 7124442 + 0.4244 * rs 76327806 - 0.0030 * rs 6265 - 0.0017 * rs 12273539 + 0.0551 * rs 11030104 - 0.1257 * rs 12291186 + 0.3375 * rs 55848362 + 0.0458 * rs 72878196 + 0.3838 * rs 10835211 + 0.1721 * rs 16917237 - 0.2612 * rs 73446388 + 0.0966 * rs 12293082 + 0.0459 * rs 4923468 - 0.7055 * rs 11030119 + 0.1402 * rs 76368953 + 0.0463 * rs 72881263 + 0.2426 * rs 80083564 - 0.0111 * rs 74435097 + 0.0505 * rs 10219241 - 0.0088 * rs 80128513 - 0.7775 * rs 28383487 + 0.6898 * rs 55958405 + 0.8550 * rs 28431965 + 0.7526 * rs 114913258 ,$$

where the value for rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258, respectively, is 0, 1, or 2, depending on the number of the patient's minor allele (see Table 1).

### Example 3—the TAK-315 Trial

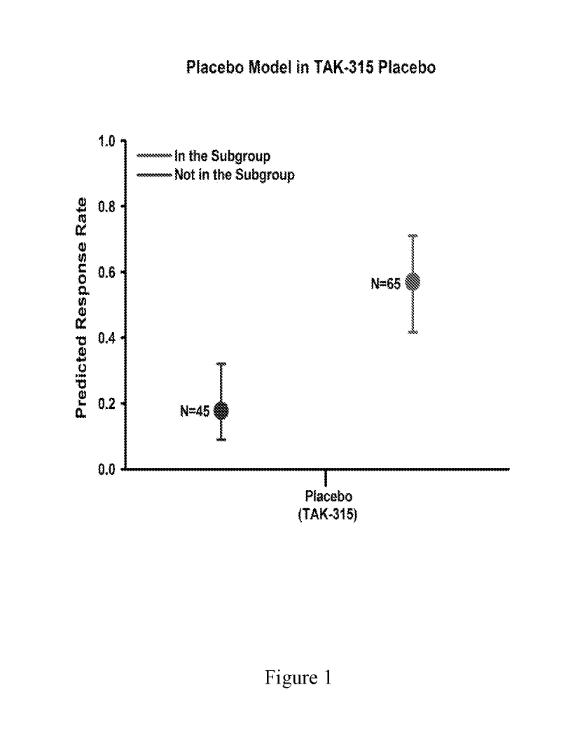

Using association testing within the placebo arm, the 24-SNP model showed statistically significant evidence for placebo effect in the TAK-315 Trial. FIG. 1 shows the Predicted Response Rate (LSmean) for the MADRS score associated with the 24-SNP model in the TAK-315 Trial. The 24 variants significantly defined a subgroup size of 59.1% within the placebo arm that showed statistical evidence with a bootstrap adjusted P-value of 8.39E-5 of a higher MADRS response. The mean response rate in the sugroup [N=65; BM (+)] was 56.98% (CL: 0.4160-0.7111) and not in the subgroup [N=45]; BM (−)] was 17.67% (CL: 0.0890-0.3202).

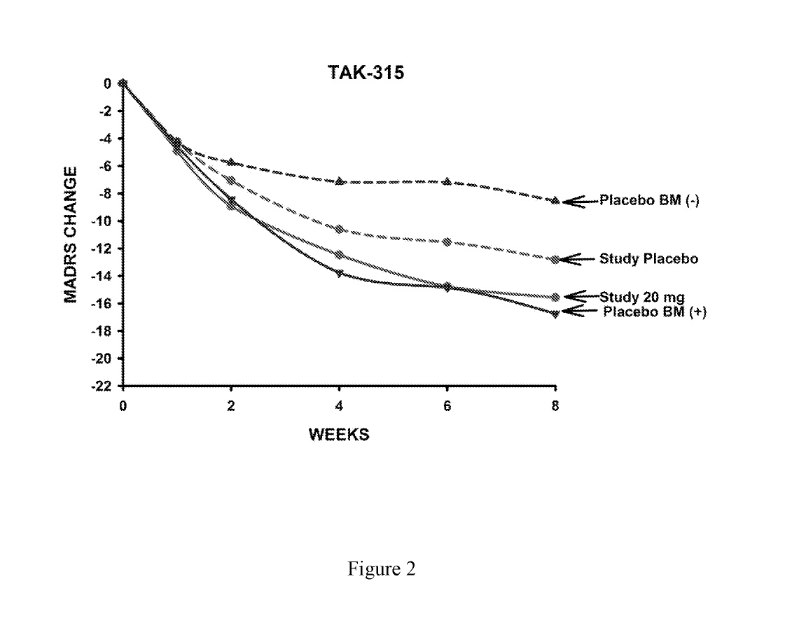

FIG. 2 shows changes from baseline in MADRS scores from the TAK-315 Trial after treatment with 20 mg vortioxetine (solid red line with circles) vs. placebo (dashed red line with circles) and the 24-SNP Placebo BM (+) subgroup (solid blue line with triangles) and not in the Placebo subgroup, BM (−) (dashed blue line with triangles). The LS mean [Std. Err. (SE)] for Placebo BM(+) was −15.68 (1.29). The LS mean for active 20 mg drug was −15.56 (1.67) with a P-value (active vs. placebo BM(+)) of 0.995. The LS mean difference was 0.12 (SE: 2.11 with 95% CL−4.05-4.29). The robust Placebo response and the similarity of the Placebo BM(+) to the study (Vortioxetine 20 mg) response is indicative of a strong placebo response.

### Example 4—the TAK-316 Trial

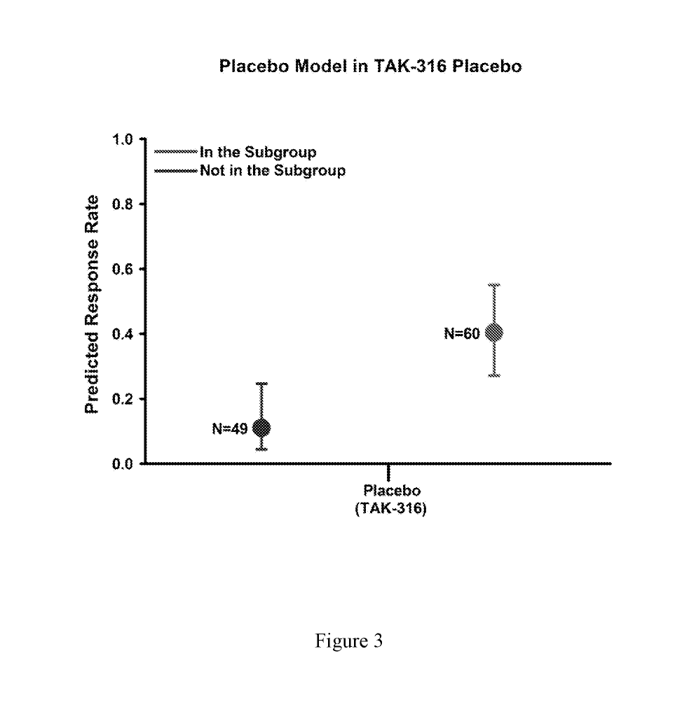

Using association testing within the placebo arm, the 24-SNP model showed statistically significant evidence for placebo effect in the TAK-316 Trial. FIG. 3 shows Predicted Response Rate (LS mean) for the MADRS score associated with the 24-SNP model in the TAK-316 Trial. The 24 variants significantly defined a subgroup size of 55.0% within the placebo arm that showed statistical evidence with a bootstrap adjusted P-value of 0.0006 of a higher MADRS response. The mean response rate in the subgroup [N=60; BM(+)] was 40.30% (CL: 0.2713-0.5504) and not in the subgroup [N=49; BM(−)] was 10.96% (CL: 0.0443-0.2464).

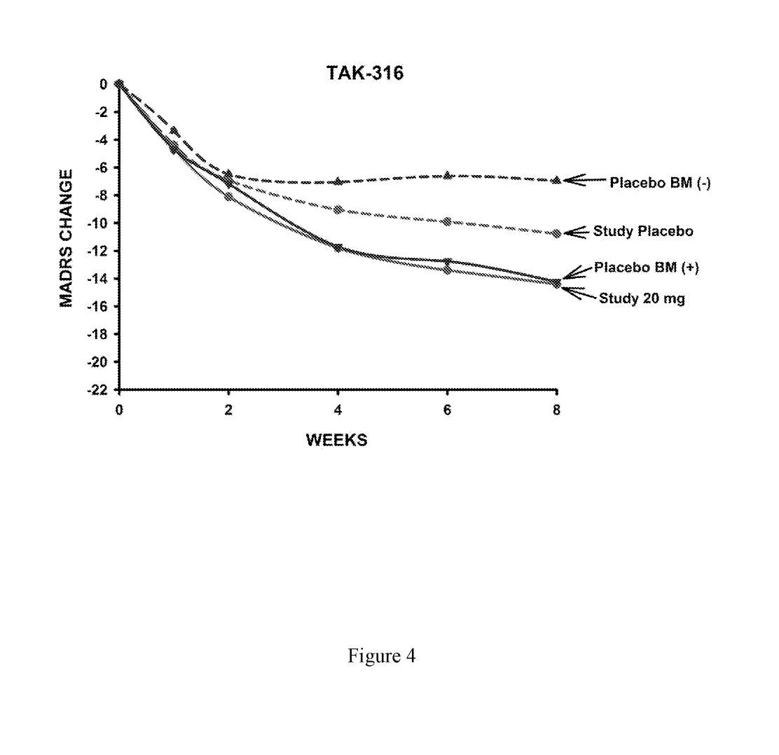

FIG. 4 shows changes from baseline in MADRS scores from the clinical TAK-316 trial after treatment with 20 mg vortioxetine (solid red line with circles) vs. placebo (dashed red line with circles) and the 24-SNP Placebo BM(+) subgroup (solid blue line with triangles) and not in the Placebo subgroup (BM(−), dashed blue line with triangles). The LS mean (SE) for Placebo BM(+) was −13.98 (1.38). The LS mean for active 20 mg drug was −13.87 (1.63) with a P-value (active vs. placebo BM(+)) of 0.960. The LS mean difference was 0.12 (SE:2.14) with 95% CL−4.13-4.35. The robust Placebo response and the similarity of the Placebo BM(+) to the study (Vortioxetine 20 mg) response is indicative of a strong placebo response.

### Example 5—the TAK-317 Trial

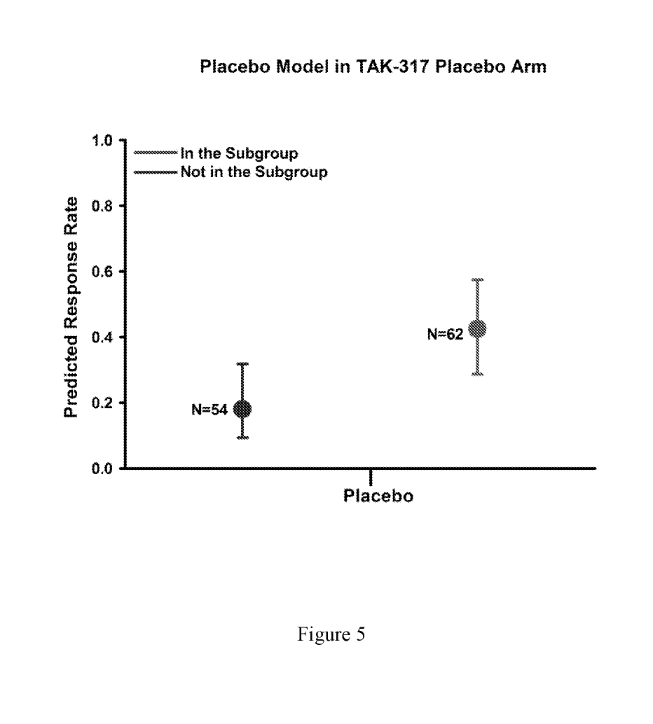

Using association testing within the placebo arm, the 24-SNP model showed statistically significant evidence for placebo effect in the TAK-317 Trial. FIG. 5 shows the Predicted Response Rate (LS mean) for the MADRS score associated with the 24-SNP model in the TAK-317 Trial. The 24 variants significantly defined a subgroup size of 53.4% within the placebo arm that showed statistical evidence with a bootstrap adjusted P-value of 0.0073 of a higher MADRS response. The mean response rate in the subgroup [N=62; BM(+)] was 42.42% (CL: 0.2864-0.5750) and not in the subgroup [N=54; BM(−)] was 17.96% (CL: 0.0933-0.3179).

### Example 6—Combined TAK-315 and TAK-316 Trials

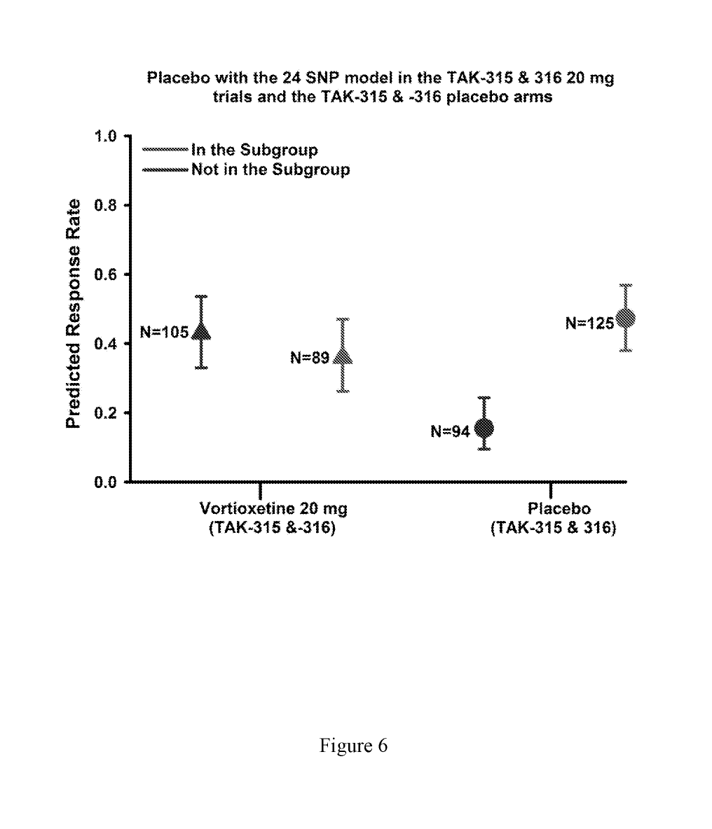

Using association testing within the placebo arm, the 24-SNP model showed statistically significant evidence for placebo effect in the combined data from the TAK-315 and TAK-316 Trials (both were 20 mg Vortioxetine trials). FIG. 6 shows the Predicted Response Rate (LSmean) for the MADRS score associated with the 24-SNP model (from the TAK-315, -316, and -317 placebo arms) in the TAK-315/316 Trials. The 24 variants significantly defined a subgroup size of 57.1% within the placebo arm that showed statistical evidence with a treatment by subgroup interaction bootstrap adjusted P-value of 1.8E-5 of a higher MADRS response. In the placebo arm, the mean response rate in the subgroup [N=125; BM(+)] was 47.33% (CL: 0.38-0.57) and not in the subgroup [N=94; BM(−)] was 15.52% (CL: 0.1-0.25). The subgroup size in the the placebo arm was 57.1% [BM(+)] and 42.9% [BM(−)]. In the Vortioxetine arm, the mean response rate for the placebo BM(+) was 35.96% (CL: 0.26-0.47) and placebo BM(−) 43.0% (CL: 0.33-0.54). The placebo subgroup size in the Vortioxetine 20 mg treat arm was 45.9(%) [BM(+), N=89] and 54.1% [BM(−), N=105].

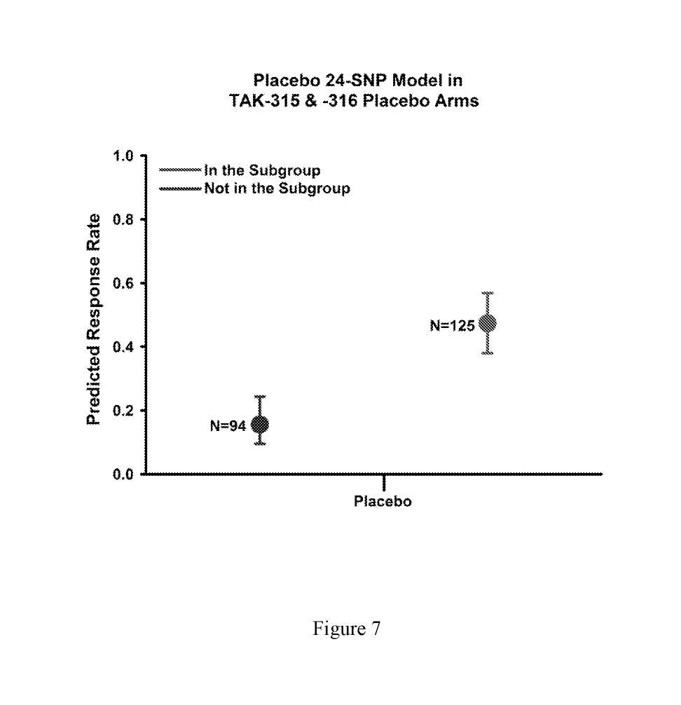

FIG. 7 shows the Predicted Response Rate (LSmean) for the MADRS score associated with the 24-SNP model in the TAK-315/316 Trials in the placebo arms. The 24 variants significantly defined a subgroup size of 57.1% within the placebo arm that showed statistical evidence with a bootstrap adjusted P-value of 2.39.E-7 of a higher MADRS response. The mean response rate in the subgroup [N=125; BM (+)] was 48.33% (CL: 0.3831-0.5848) and not in the subgroup [N=94; BM (−)] was 15.06% (CL: 0.0896-0.2421). The subgroup size in the the placebo arm was 57.1% [BM (+)] and 42.9% [BM(−)].

### Example 7—Combined TAK-315, TAK-316 and TAK-317 Trials

Using association testing within the placebo arm, the 24-SNP model showed statistically significant evidence for placebo effect when taking into account each of the TAK-315, 316, and 317 Trials, as shown in Table 3.

TABLE 3
Results for association testing within Placebo arm

| | | | rs28431965 | | | |
|---|---|---|---|---|---|---|
| | | | (chr18: 60947535; | | rs114913258 | |
| | | | BCL2; tier 2) | | (chr1: 20760566; | |
| | BDNF (chr 11; tier 1) | | | Treat. | intergenic; tier 3) | |
| | 33 less common and rare variants | | | Effect | | Treat. |
| | Main Effect | Treat. Effect | Main Effect w/in | PBO vs. | Main Effect w/in | Effect PBO |
| Outcome | w/in PBO Arms | PBO vs. Vorti | PBO Arms | Vorti | PBO Arms | vs. Vorti |
| Response | 0.0042/0.0463 | 0.102467 | 1.38E−04/1 | 0.005268 | 1.14E−04/1 | 0.001791 |
| HAM-A | 0.0195/0.2142 | 0.125428 | 1.61E−06/0.0178 | 0.000191 | 1.02E−05/1 | 0.000381 |
| MADRS | 0.0026/0.0291 | 0.034924 | 2.45E−07/0.0027 | 0.000346 | 4.66E−08/0.0786 | 2.98E−05 |

Treat. = treatment;
PBO = Placebo;
Vorti. = Vortioxetine;
MADRS = Montgomery Åsberg Depression Rating Scale;
HAM-A—Hamilton Anxiety Scale score;
Listed values are uncorrected P value/Bonferroni corrected P value.
If only a single value is listed, it represents an uncorrected P value.

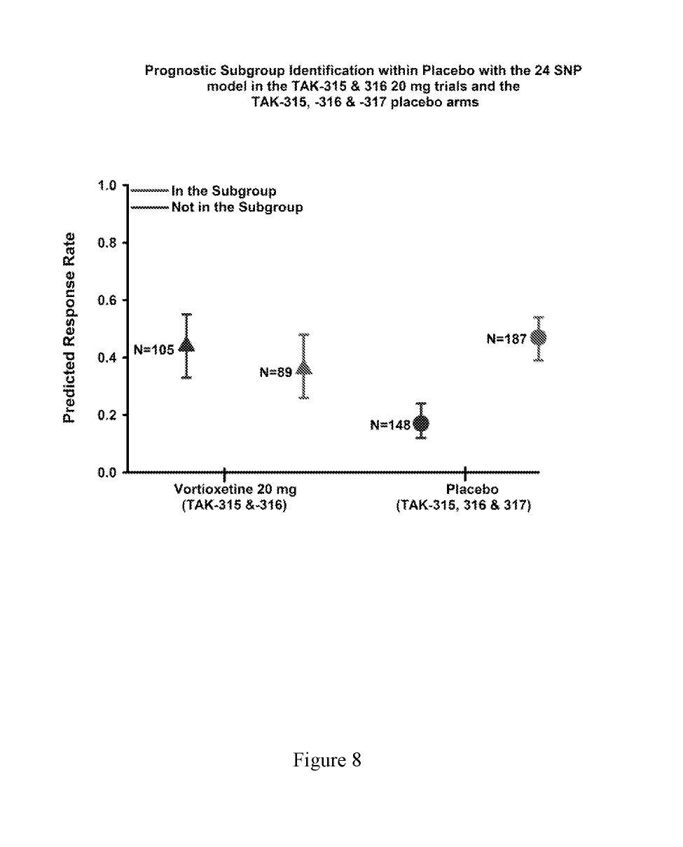

FIG. 8 provides a comparison of the 24 SNP placebo model in the three trials' (TAK-315, 316 & 317) placebo arms and the 20 mg vortioxetine dose in TAK-315 and -316. The plot shows the Predicted Response Rate [LS with 95% Confidence Level mean (LSmean)] for the MADRS score associated with the 24-SNP model. The 24 variants significantly defined a subgroup size of 55.82% within the placebo arm with an Odds Ratio (OR) in the placebo arm of 4.08 [Confidence Level (CL) 2.42-6.85] that showed statistical evidence with a treatment by subgroup interaction bootstrap adjusted P-value of 0.0126 of a higher MADRS response. In the placebo arm the mean response rate in the subgroup [N=187; BM(+)] was 46.63% (CL: 0.39-0.55) and not in the subgroup [N=148; BM(−)] was 17.02% (CL: 0.12-0.24). The subgroup size in the the placebo arm was 55.8% [BM(+)] and 44.2% [BM(−)]. In the Vortioxetine arm, the mean response rate for the placebo BM(+) was 36.00% (CL: 0.26-0.48) and placebo BM(−) was 44.0% (CL: 0.33-0.55). The subgroup size in the Vortioxetine 20 mg treat arm was 45.9% [BM(+), N=89] and 54.1% [BM(−), N=105].

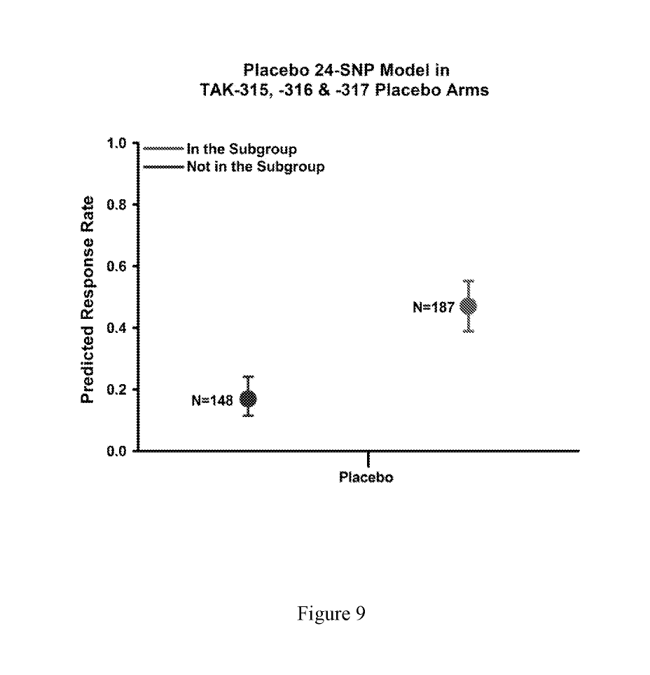

FIG. 9 shows the Predicted Response Rate (LSmean) for the MADRS score associated with the 24 SNP placebo model in the three trials (TAK-315, 316 & 317) placebo arms. The 24 variants significantly defined a subgroup size of 55.8% within the placebo arm that showed statistical evidence with a bootstrap adjusted P-value of 8.10E-9 of a higher MADRS response. The mean response rate in the subgroup [N=187; BM(+)] was 46.89% (CL: 0.3882-0.5513) and not in the subgroup [N=148]; BM(−)] was 16.80% (CL: 0.1142-0.24030). The subgroup size in the the placebo arm was 55.8% [BM(+)] and 44.2% [BM(−)].

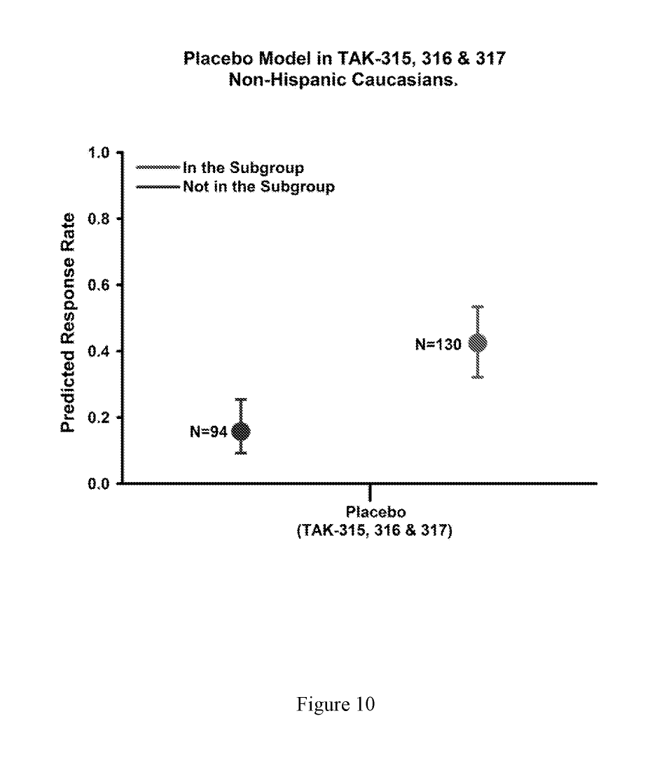

FIG. 10 shows the Predicted Response Rate (LS means) for the MADRS score associated with the 24-SNP model in Non-Hispanic Caucasians from the placebo arms of the TAK-315, 316, and 317 Trials. The 24 variants significantly defined a subgroup size of 58.04% within the placebo arm that showed statistical evidence with a bootstrap adjusted P-value of 1.31E-5 of a higher MADRS response. The mean response rate in the subgroup [N=130; BM(+)] was 42.43% (CL: 0.3212-0.5345) and not in the subgroup [N=94; BM(−)] was 15.69% (CL: 0.0921-0.2545). The subgroup size in the placebo arm was 58.0% [BM(+)] and 42.055 [BM(−)]

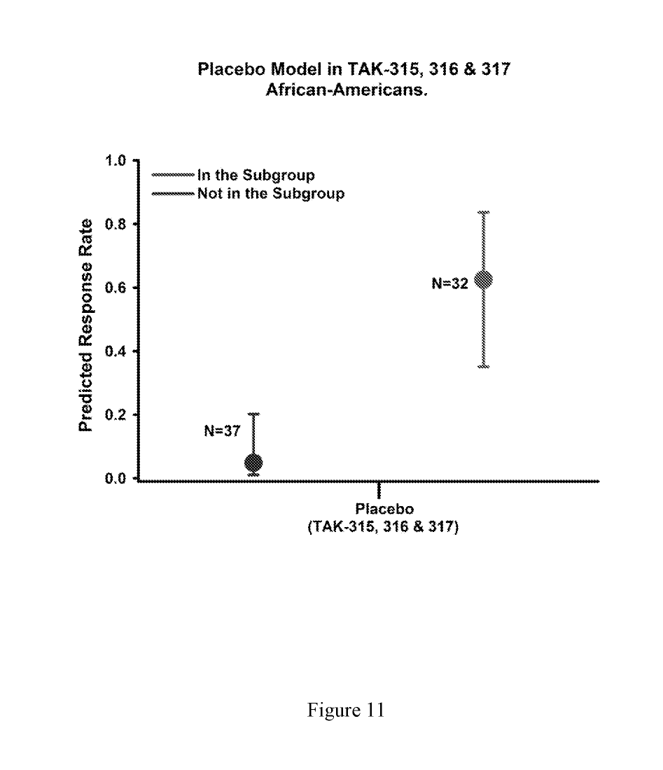

FIG. 11 shows the Predicted response Rate (LS means) for the MADRS score associated with the 24-SNP model in African Americans from the placebo arms of the TAK-315, 316, and 317 Trials. The 24 variants significantly defined a subgroup size of 46.40% within the placebo arm that showed statistical evidence with a bootstrap adjusted P-value of 1.48E-5 of a higher MADRS response. The mean response rate in the subgroup [N=32; BM(+)] was 62.43% (CL: 0.3504-0.8366) and not in the subgroup [N=37; BM(−)] was 4.85% CL: 0.0102-0.2016). The subgroup size in the placebo arm was 46.4% [BM(+)] and 53.6% [BM(−)].

What is claimed is:

1. A method for treating depression and/or a major depressive disorder (MDD) in an individual, comprising administering a placebo to an individual identified as (i) BDNF variant positive, (ii) BCL2 variant positive, and/or intergenic variant positive.

2. The method of claim 1, wherein the individual suffers from a MDD.

3. The method of claim 1, wherein the individual is homozygous for a BDNF variant and/or a BCL2 variant and/or an intergenic variant.

4. The method of claim 1, wherein the individual is heterozygous for a BDNF variant and/or a BCL2 variant and/or an intergenic variant.

5. The method of claim 1, wherein the individual is BDNF variant positive, BCL2 variant positive, and intergenic variant positive.

6. The method of claim 1, wherein the BDNF variant is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

7. The method of claim 1, wherein the BCL2 variant is rs28431965.

8. The method of claim 1, wherein the intergenic variant is rs114913258.

9. The method of claim 5, wherein the individual has rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258 variants.

10. A method for determining the likelihood that an individual suffering from depression and/or MDD will experience an enhanced placebo effect when treated with a placebo comprising: assaying a biological sample from the individual for the presence or absence of a BDNF variant and/or a BCL2 variant and/or an intergenic variant.

11. The method of claim 10, wherein the individual has a clinical diagnosis of a MDD.

12. The method of claim 10, wherein the BDNF variant is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

13. The method of claim 10, wherein the BCL2 variant is rs28431965.

14. The method of claim 10, wherein the intergenic variant is rs114913258.

15. The method of claim 10, wherein the individual has rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258 variants.

16. The method of claim 10, wherein the sample is selected from the group consisting of a body fluid sample, a tissue sample, cells and isolated nucleic acids.

17. The method of claim 10, wherein the isolated nucleic acids comprise DNA.

18. The method of claim 10, wherein the isolated nucleic acids comprise RNA.

19. The method of claim 10, wherein the assaying comprises reverse transcribing the RNA to produce cDNA.

20. The method of claim 10, comprising detecting the presence of a BDNF variant and/or a BCL2 variant and/or an intergenic variant in nucleic acids from the individual.

21. The method of claim 10, wherein the individual is homozygous for the BDNF variant and/or the BCL2 variant and/or the intergenic variant.

22. The method of claim 10, wherein the individual is heterozygous for the BDNF variant and/or the BCL2 variant and/or the intergenic variant.

23. A method for determining the likelihood that an individual suffering from depression and/or MDD will respond favorably to administration of a placebo comprising: assaying a biological sample from the individual for the presence of a BDNF variant and/or a BCL2 variant and/or an intergenic variant.

24. The method of claim 23, wherein the individual has a clinical diagnosis of MDD.

25. The method of claim 23, wherein the BDNF variant is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

26. The method of claim 23, wherein the BCL2 variant is rs28431965.

27. The method of claim 23, wherein the intergenic variant is rs114913258.

28. The method of claim 23, wherein the individual has rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258 variants.

29. The method of claim 23, wherein the biological sample is selected from the group consisting of a body fluid sample, a tissue sample, cells and isolated nucleic acids.

30. The method of claim 29, wherein the isolated nucleic acids comprise DNA.

31. The method of claim 29, wherein the isolated nucleic acids comprise RNA.

32. The method of claim 23, wherein the assaying comprises reverse transcribing RNA to produce cDNA.

33. The method of claim 23, wherein the assaying comprises nucleic acid sequencing.

34. The method of claim 23, wherein the individual is homozygous for the BDNF variant and/or the BCL2 variant and/or the intergenic variant.

35. The method of claim 23, wherein the individual is heterozygous for the BDNF variant and/or the BCL2 variant and/or the intergenic variant.

36. A kit comprising: (i) at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405, rs28431965, and rs114913258, and (ii) a detectably labeled probe that hybridizes to the genetic variant.

37. The kit of claim 36, wherein the kit comprises: at least one pair of primers that specifically hybridizes to a genetic variant independently selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487 and rs55958405; a pair of primers that specifically hybridizes to rs28431965; and a pair of primers that specifically hybridizes to rs114913258.

38. A method for identifying active agent responders in a clinical trial for treating depression and/or MDD, comprising excluding from the clinical trial an individual identified as (i) BDNF variant positive, (ii) BCL2 variant positive, and/or (iii) intergenic variant positive.

39. The method of claim 38, wherein the individual suffers from MDD.

40. The method of claim 38, wherein the individual is homozygous for a BDNF variant and/or a BCL2 variant and/or an intergenic variant.

41. The method of claim 38, wherein the individual is heterozygous for a BDNF variant and/or a BCL2 variant and/or an intergenic variant.

42. The method of claim 38, wherein the individual is BDNF variant positive, BCL2 variant positive, and intergenic variant positive.

43. The method of claim 38, wherein the BDNF variant is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

44. The method of claim 38, wherein the BCL2 variant is rs28431965.

45. The method of claim 38, wherein the intergenic variant is rs114913258.

46. The method of claim 42, wherein the individual has rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258 variants.

47. A method for identifying active agent responders in a clinical trial for treating depression and/or MDD, comprising excluding from data analysis data collected from an individual identified as (i) BDNF variant positive, (ii) BCL2 variant positive, and/or (iii) intergenic variant positive in the clinical trial.

48. The method of claim 47, wherein the individual suffers from a MDD.

49. The method of claim 47, wherein the individual is homozygous for a BDNF variant and/or a BCL2 variant and/or an intergenic variant.

50. The method of claim 47, wherein the individual is heterozygous for a BDNF variant and/or a BCL2 variant and/or an intergenic variant.

51. The method of claim 47, wherein the individual is BDNF variant positive, BCL2 variant positive, and intergenic variant positive.

52. The method of claim 47, wherein the BDNF variant is selected from the group consisting of rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, and combinations thereof.

53. The method of claim 47, wherein the BCL2 variant is rs28431965.

54. The method of claim 47, wherein the intergenic variant is rs114913258.

55. The method of claim 51, wherein the individual has rs7124442, rs76327806, rs6265, rs12273539, rs11030104, rs12291186, rs55848362, rs72878196, rs10835211, rs16917237, rs73446388, rs12293082, rs4923468, rs11030119, rs76368953, rs72881263, rs80083564, rs74435097, rs10219241, rs80128513, rs28383487, rs55958405, rs28431965, and rs114913258 variants.


SEQUENCE LISTING (24 sequences)

SEQ ID NO: 1 - DNA, 51 bp, Homo sapiens
aaggaagctgcataaagttgacatayagcagatattccaagcattccttac

SEQ ID NO: 2 - DNA, 51 bp, Homo sapiens
atacgagtgtcatgatgtgacacaaygtgttcacttgttcacagcagtggt

SEQ ID NO: 3 - DNA, 51 bp, Homo sapiens
tcctcatccaacagctcttctatcaygtgttcgaaagtgtcagccaatgat

SEQ ID NO: 4 - DNA, 51 bp, Homo sapiens
actcaatgcttcatcacttctgctcygatcaggacagagtccttggagtgc

SEQ ID NO: 5 - DNA, 51 bp, Homo sapiens
attaaaaagcagataacactaccacrtactaactgtcctacaatttcctgt

SEQ ID NO: 6 - DNA, 51 bp, Homo sapiens
agaagacatgcaaactcaaataccamcatatttcatctaagcataggacag

SEQ ID NO: 7 - DNA, 51 bp, Homo sapiens
actagagctggcgcaagcccatggcyatggtgaggcagcgtttccactgga

SEQ ID NO: 8 - DNA, 51 bp, Homo sapiens
ctttgctttaggaaaatcattttcamggttgcttcatgcaaagtgggaata

SEQ ID NO: 9 - DNA, 51 bp, Homo sapiens
ttcctgtttcaccagcagagctctgrtcgctcagttgaagctgaaagtcag

SEQ ID NO: 10 - DNA, 51 bp, Homo sapiens
ttcctaccaccattacatacttctgktaatcaattatcttccttctcccct

SEQ ID NO: 11 - DNA, 51 bp, Homo sapiens
aggaaagaaggagactggcctcgtcycacaactttggggtgggggatcccc

SEQ ID NO: 12 - DNA, 51 bp, Homo sapiens
aattgtaccagtgccttgcatggaaytctaaatattattttttatttgctt

SEQ ID NO: 13 - DNA, 51 bp, Homo sapiens
cttgaaaaaataatgctacctatttmacttttgtggggcttaaaaattaat

SEQ ID NO: 14 - DNA, 51 bp, Homo sapiens
ttaagtcaccactcagacttttctcrtagcaaaagatcagatctcacaacc

SEQ ID NO: 15 - DNA, 51 bp, Homo sapiens
gagatgatttaggtgatttcaatgcrtgaagggaatcagtgaactagatca

SEQ ID NO: 16 - DNA, 51 bp, Homo sapiens
caaacttatgaaatagcttctaaacrtactaaatcctaatccaaacaattt

SEQ ID NO: 17 - DNA, 51 bp, Homo sapiens
agatggggaggcacaacttcataatkgactttggaaccagaaaactttggg

SEQ ID NO: 18 - DNA, 51 bp, Homo sapiens
cttaatttcccctacactgcaagaartagttatagactatatcttttatat

SEQ ID NO: 19 - DNA, 51 bp, Homo sapiens
aactttccattaggcctgaaattttraaaactatttgtaaaaaatgttttt

SEQ ID NO: 20 - DNA, 51 bp, Homo sapiens
ctccagccccgatctcagtgtgagcmgaacctcagaaaagacgctttttaa

SEQ ID NO: 21 - DNA, 51 bp, Homo sapiens
cttgtcaggctagggcgggaagacckctggggaacttgttgcttatcagcg

SEQ ID NO: 22 - DNA, 51 bp, Homo sapiens
tatatttttctgactcttttgtcaamagtttgtggtctgtatagaaactat

SEQ ID NO: 23 - DNA, 51 bp, Homo sapiens
ctttcaaagcctcagtttccgtatcyattaaatgaaggtgataatatctat

SEQ ID NO: 24 - DNA, 51 bp, Homo sapiens
tctgtctcctctgccagtctgagagytcctctgggacagggtacttggtct